(19) United States
(12) Patent Application Publication
(10) Pub. No.: US 2009/0254083 A1
(43) Pub. Date: Oct. 8, 2009

(54) ROBOTIC ABLATION CATHETER

(75) Inventors: Daniel T. Wallace, Santa Cruz, CA (US); Katherine Whitin Lee, Los Altos, CA (US); Sean Murphy, Fremont, CA (US); Randall L. Schlesinger, San Mateo, CA (US); Eric A. Schultheis, Los Altos, CA (US); William K. Yee, San Jose, CA (US)

Correspondence Address: VISTA IP LAW GROUP LLP, 12930 Saratoga Avenue, Suite D-2, Saratoga, CA, 95070, US

(73) Assignee: Hansen Medical, Inc., Mountain View, CA (US)

(21) Appl. No.: 12/401,588

(22) Filed: Mar. 10, 2009

Related U.S. Application Data
(60) Provisional application No. 61/068,911, filed on Mar. 10, 2008.
(60) Provisional application No. 61/127,042, filed on May 8, 2008.

(51) Int. Cl. A61B 18/14

(52) U.S. Cl. 606/41

(57) ABSTRACT

Assemblies, systems, and methods related to remotely-steerable ablation procedures are described. A necked-down ablation catheter may be coupled within a working lumen of a robotically-steerable sheath configured to be driveably coupled to an electromechanical instrument driver. The ablation catheter may be an irrigated ablation catheter having an irrigation fluid reservoir at its distal tip. The outer diameter of the distal portion of the ablation catheter is generally larger than that of the more proximal aspects due, in part, to the fact that the proximal aspects are designed to fit through a relatively low-profile steerable sheath.

### FIELD OF THE INVENTION

The invention relates generally to remotely controlled medical devices and systems, such as telerobotic surgical systems or manually steerable catheters, and the employment thereof for conducting procedures involving ablation of tissues, such as endocardial tissues. More particularly, this invention relates to systems, apparatuses, and methods for conducting electrophysiologic procedures with instruments having optimized geometric and thermal properties.

### BACKGROUND

In certain electrophysiological (“EP”) procedures, more than one elongate instrument may be combined, e.g., in a tandem fashion, and operated as an assembly. Referring to FIG. 1A, a conventional EP mapping and/or ablation catheter (2), such as those available from Boston Scientific or the Biosense Webster division of Johnson & Johnson, is depicted. The depicted EP catheter (2) has a substantially flexible and generally tubular body (3), a distal end (5) that generally comprises a distal tip electrode (4) and a relatively proximal tip electrode (6), a proximal interface (9) having a steering handle (8), and a proximal electronics interface (10) for interfacing with equipment to read signals coming from the distal tip electrodes (4, 6), among other things. The outer diameter (12) of the distal tip may be approximately equivalent to the outer diameter of the flexible body (3), although the tip electrodes may protrude slightly outward from the surface to facilitate contact between the electrodes (4, 6) and surfaces of tissue structures. Alternatively, the electrodes (4, 6) may be recessed slightly from the surface to provide a slight offset from direct contact with the tissue structures. The tubular body (3) may or may not be completely uniform. When employing a conventional EP catheter (2) such as that depicted in FIG. 1A, it may be desirable to forgo the steering functionality provided by the manually-operated handle (8) in favor of a steerable sheath instrument through which the EP catheter (2) may be placed. In other words, given an EP catheter of limited navigability and a highly-steerable and controllable sheath through which such EP catheter may be placed and thereby navigated with precision, the assembly comprising both the conventional EP catheter and the highly-steerable sheath may be selected. Referring to FIG. 1B, a robotic sheath instrument (14), such as those described in U.S. patent application Ser. Nos. 10/923,660, 10/949,032, 11/073,363, 11/173,812, 11/176,954, 11/179,007, 11/176,598, 11/176,957, 11/185,432, 11/202,925, 11/331,576, 11/418,398, 11/481,433, 11/637,951, 11/640,099, 11/678,001, 11/678,016, 60/919,015, 11/690,116, 60/920,328, 60/925,449, 60/925,472, 60/926,060, 60/927,682, 11/804,585, 60/931,827, 60/934,639, 60/934,688, 60/961,189, 11/762,778, 11/762,779, 60/961,191, 11/829,076, 11/833,969, 60/962,704, 60/964,773, 60/964,195, 61/068,911, 11/852,255, 11/906,746, 61/003,008, 11/972,581, 12/022,987, 12/024,883, 12/024,760, 12/024,642, 12/032,626, 12/032,634, 12/032,622, 12/032,639, and 12/012,795, each of which is incorporated by reference in its entirety into this disclosure, is depicted. The catheter body (24) defines a through-lumen (18) which is sized to accept instruments such as EP catheters. The robotic sheath instrument (14) has a proximal end (20), a distal end (22), and a proximal interface assembly (16) for mechanically associating or operatively coupling with an electromechanical instrument driver (not shown) for driving or steering the robotic sheath instrument (14), such as the catheter body (24). Referring to FIG. 1C, a manually steerable sheath instrument (26) is depicted. The steerable sheath instrument (26) includes a tubular body (30) that defines a through lumen (28) which is sized to accept various medical instruments or tools. A steering handle (32) provides manual steering of the sheath (26), such as the tubular body (30). Referring to FIG. 1D, an assembly comprising a first robotic sheath instrument (14) may be placed or disposed through or into a second robotic sheath instrument (34) is depicted, as described in the aforementioned incorporated disclosures. The second robotic sheath (34) has a tubular body (38) which includes a proximal end (40), a distal end (42), and a through lumen (44) that is sized to accept the first robotic sheath (14), which may have a smaller tubular body (24). The second robotic sheath instrument (34) also has a proximal interface assembly (36) for mechanically interfacing with a robotic instrument driver (not shown) to drive or steer the second sheath instrument (34), such as the tubular body (38). Similarly, the first robotic sheath instrument (14) has a tubular body (24) that includes a proximal end (20), a distal end (22), and a through-lumen (18) through which other medical instruments may be passed and applied to perform various medical procedures. The first robotic sheath instrument (14) also has a proximal interface assembly (16) for mechanically interfacing with the robotic instrument driver (not shown) to controllably bend or steer aspects of the first sheath instrument (14), such as the tubular body (24). Each of the assemblies in FIGS. 1B, 1C, and 1D may be used to assist in the steering of a conventional EP catheter (2), such as that depicted in FIG. 1A when the EP catheter (2) is placed through a working lumen of the larger steerable sheath instrument. For example, referring to FIG. 1E, a robotically-steerable catheter (14), such as that illustrated in FIG. 1B, is depicted with a conventional EP catheter (2) placed through the working lumen (18) of the sheath body (24) of the larger steerable sheath instrument (14). The distal tip (5) of the EP catheter (2) protrudes out past the distal end (22) of the sheath body (24) and may be used for EP diagnostic and/or interventional procedures. It may be desirable to oscillate the EP catheter (2) and robotic sheath instrument (14) relative to each other (99), for example, to facilitate the sensing of forces applied at the distal end (5) of the EP catheter (2), as described in the aforementioned incorporated applications, and thus freedom of relative axial and/or rotational motion along the longitudinal axes of these instruments may be desirable.

FIG. 1F illustrates a variation similar to that depicted in FIG. 1E, with the exception that an irrigated EP catheter (2A) having an irrigated tip (5A) is placed through the working lumen (18) of the sheath body (24) of the sheath instrument (14). The distal tip (5A) of the irrigated ablation catheter (2A) protrudes out past the distal end (22) of the sheath body (24) and may be used for EP diagnostics and/or intervention procedures. Similar to the EP catheter illustrated in FIG. 1A, the irrigated ablation catheter (2A) includes electrodes 4A and 6A. In one version of the catheter (2A), the irrigation system may be a so-called “closed-loop irrigation” configuration, such as that illustrated in close-up sectional view in FIG. 1G. Referring to FIG. 1G, with a closed-loop configuration, coolant may be pumped and flowed down channels (52, 54) and returned proximally through a return channel (56) to a coolant circulation subsystem, such as a pump (not shown), before it is again circulated through the cooling channels (52, 54). Another variation of an irrigated catheter (2A) may comprise a so-called “open-loop irrigation” configuration such as that illustrated in close-up sectional view in FIG. 1H. Referring to FIG. 1H, with an open-loop configuration, coolant, such as saline, may be pumped and flowed distally through channels (52, 54) and out of an exit port (58) without a proximal return.

One of the potential downsides of selecting a combination of instruments, such as an ablation catheter instrument placed through a working lumen of a highly-steerable robotic sheath instrument, is the net overall geometry of the assembly. Generally, it is desirable in minimally invasive diagnosis and intervention to minimize the size of instrumentation, while also retaining functionality and performance. To address such challenges in electrophysiology, specialized configurations of irrigated and non-irrigated EP catheters combined with remotely steerable sheath embodiments are presented.

### SUMMARY

One embodiment is directed to an ablation instrument system having a robotically-steerable sheath defining a working lumen and a necked-down ablation catheter having a distal ablation tip. The proximal portions of the ablation catheter have outer diameters facilitating slidable fitting or coupling with the working lumen of the ablation catheter. The distal ablation tip, however, has an outer diameter configured to prevent such tip from entering the working lumen of the sheath. In other words, the distal ablation tip has an outer diameter greater than the inner diameter of the sheath defining the working lumen. The distal ablation tip and a tubular body of the ablation catheter may be coupled with a substantially stepwise neckdown. The distal ablation tip may have a substantially cylindrical side outer shape with a substantially flat distal face. The distal ablation tip also may have a substantially cylindrical side outer shape with a substantially hemispherical distal face, or substantially bullet-shaped distal face. Further, the distal ablation tip may have a substantially spherical outer shape. The ablation catheter may have a first irrigation lumen defined between the proximal and distal ends of the catheter. The distal tip may have a thin-shell design, wherein the sidewalls have a thickness, the distal face has a thickness, and a distal irrigation reservoir volume is defined between the sidewalls, distal face, and a proximal surface of the distal ablation tip, the reservoir volume being accessible via the first irrigation lumen. The outer diameter of the distal ablation tip may be at least four times greater than the sidewall thickness in the thin-shell design. The distal face may have a substantially uniform thickness, such as a thickness at least two times greater than the sidewall thickness, or may have an inner surface having a channeled geometry to maximize surface area of a distal face inner surface. The distal tip may further comprise a metallic heat sink member suspended in the distal irrigation reservoir volume, and this member may have a surface shape at least partially defining channels configured to maximize surface engagement between the heat sink member and fluids which may be present in the reservoir. The ablation catheter may further have a return irrigation lumen to allow at least a portion of fluid pressurized through the first irrigation lumen toward the reservoir to return to the proximal end of the ablation catheter. The ablation catheter may further have a plurality of sideholes defined through the sidewalls of the distal ablation tip to allow for the escape of irrigation fluids pressurized into the distal irrigation reservoir. The necked-down ablation catheter may also have at least one electrical lead coupled between the distal ablation tip and the proximal end of the ablation catheter, and a proximal quick-connect interface configured to allow coupling of an electrical source to the electrical lead, as well as coupling of an irrigation source to the first irrigation lumen, with manual actuation of a single mechanical coupling.

Another embodiment is directed to a method for conducting a minimally invasive ablation procedure, comprising coupling a necked-down ablation catheter through a working lumen of a robotically-steerable sheath by threading a proximal end of the ablation catheter into the lumen through a distal end of the sheath and continuing to advance the ablation catheter relative to the sheath until the proximal end of the ablation catheter at least partially emerges from a proximal end of the sheath; connecting an RF energy source and an irrigation fluid source to the proximal end of the ablation catheter; coupling at least the robotically-steerable sheath to an electromechanical instrument driver; inserting at least the distal ends of the coupled ablation catheter and robotically-steerable sheath into the patient; and utilizing the electromechanical instrument driver to navigate the distal end of the sheath and thereby navigate the distal end of the ablation catheter. Connecting an RF energy source and an irrigation fluid source to the proximal end of the ablation catheter may comprise actuating a proximal quick connect interface configured to allow coupling of the RF energy source to at least one electrical lead on the ablation catheter as well as coupling of the irrigation fluid source to a first irrigation lumen defined by the ablation catheter, with manual actuation of a single mechanical coupling. Inserting may comprise commanding the electromechanical instrument driver to insert the coupled ablation catheter and steerable sheath. The method may also include advancing irrigation fluid from a proximally disposed reservoir to a reservoir disposed within the proximal end of the ablation catheter. The method may also include disbursing at least a portion of said irrigation fluid out of the distal end of the ablation catheter through a plurality of side ports, or returning at least a portion of the advanced irrigation fluid from the distal end of the ablation catheter to the proximal end of the ablation catheter via a return irrigation lumen.

Another embodiment is directed to a method comprising inserting a distal portion of an elongate instrument into a patient's body, the elongate instrument comprising a necked down ablation catheter having a distal ablation tip, a proximal end, and a tubular body coupling the distal ablation tip and the proximal end, wherein at least a portion of the tubular body is slideably disposed through the working lumen of a robotically-steerable sheath and the outer diameter of the distal ablation tip is greater than the inner diameter of the sheath defining the working lumen; and dithering the elongate instrument relative to the sheath. The method may further comprise advancing the distal tip of the necked down ablation catheter into contact with a tissue structure surface within the patient's body and calculating loads imparted to the distal tip of the necked down ablation catheter as a result of such contact. Calculating may comprise measuring relative loads between the necked down ablation catheter and sheath during the dithering.

### BRIEF DESCRIPTION OF THE DRAWINGS

FIG. 1A illustrates a manually-steerable ablation catheter.

FIG. 1B illustrates a robotically-steerable sheath.

FIG. 1C illustrates a manually-steerable sheath.

FIG. 1D illustrates an assembly of two robotically-steerable sheaths.

FIG. 1E illustrates an assembly of a manually-steerable ablation catheter and a robotically-steerable sheath.

FIG. 1F illustrates an assembly of a manually-steerable ablation catheter and a robotically-steerable sheath.

FIG. 1G illustrates a closed-loop irrigation tip.

FIG. 1H illustrates an open-loop irrigation tip.

FIG. 2A illustrates a system in accordance with the present invention having a necked-down ablation catheter partially positioned through an inner steerable sheath, which is partially positioned through an outer steerable sheath.

FIG. 2B illustrates a method in accordance with the present invention.

FIGS. 3A-3I illustrate aspects of distal portions of necked down ablation catheters in accordance with the present invention.

FIGS. 4A-4C illustrate aspects of proximal coupling aspects of catheter configurations in accordance with the present invention.

FIGS. 5A-5B illustrate aspects of proximal coupling aspects of catheter configurations in accordance with the present invention.

FIGS. 6A-6D illustrate aspects of necked down ablation catheter embodiments in accordance with the present invention.

### DETAILED DESCRIPTION

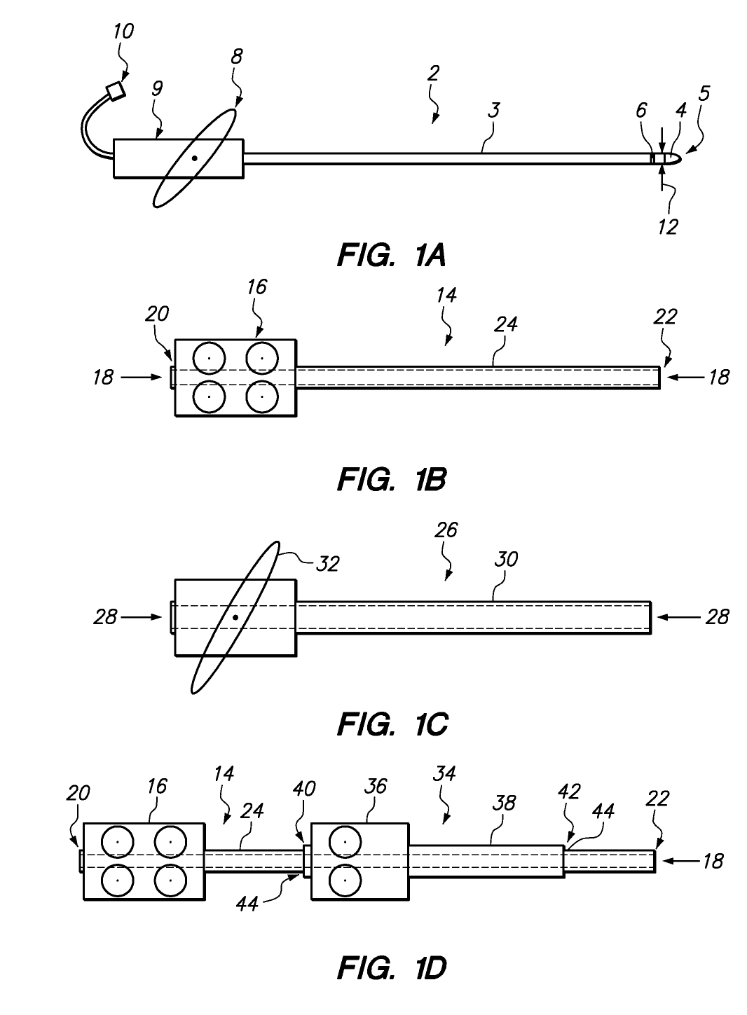

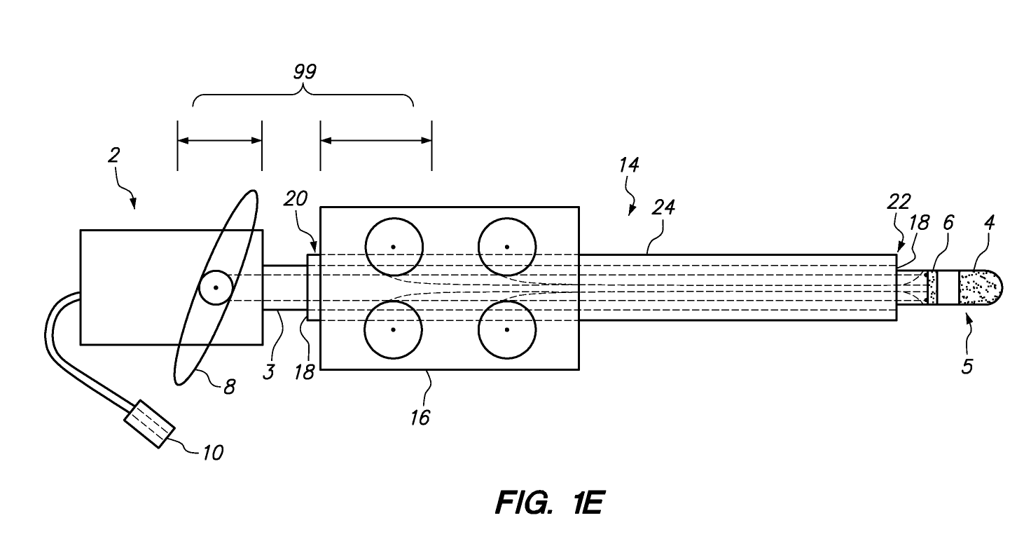

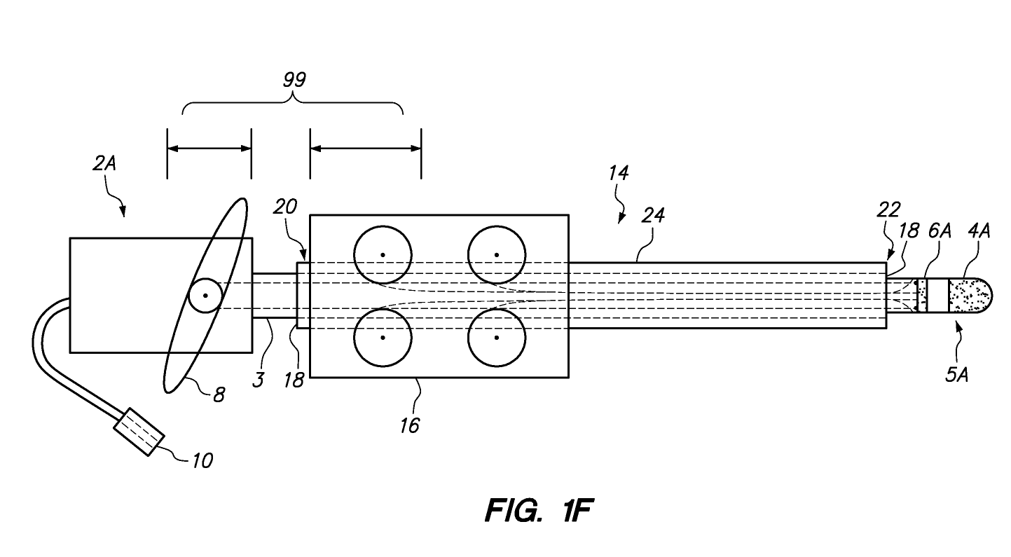

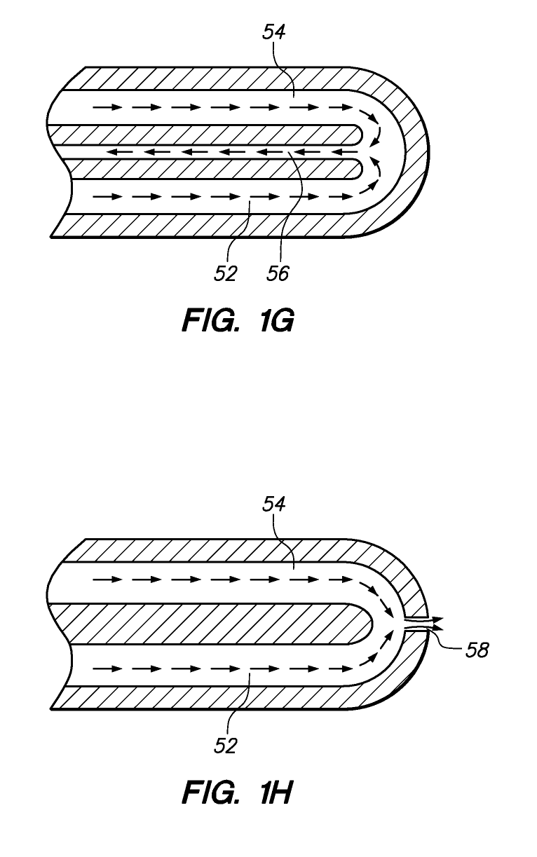

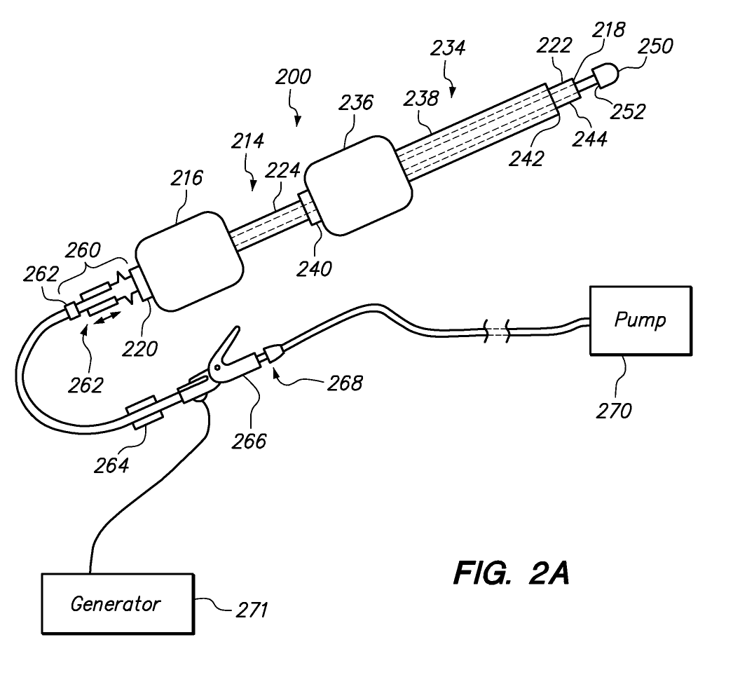

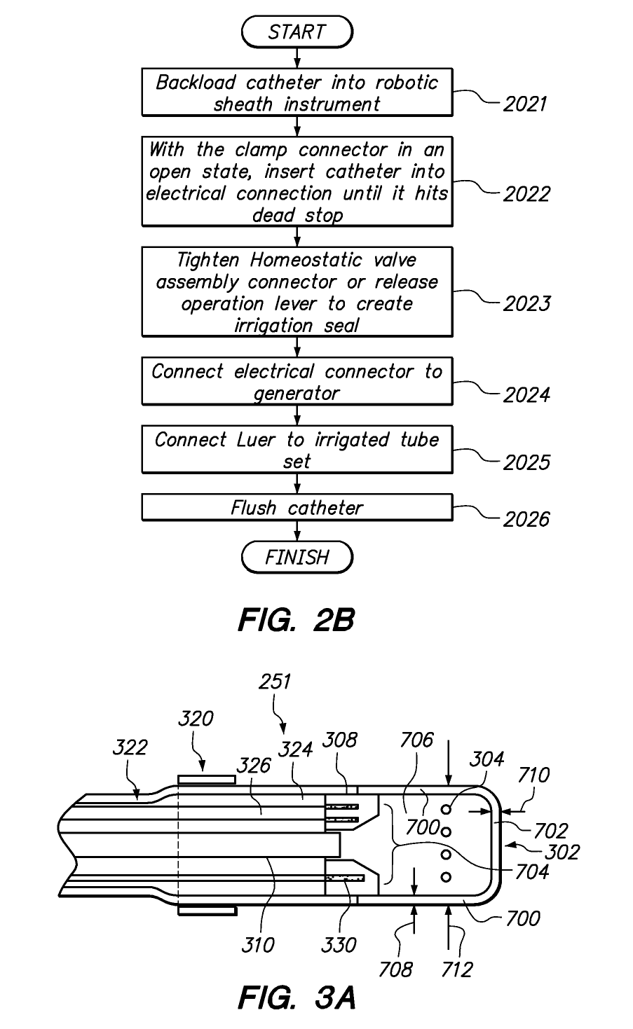

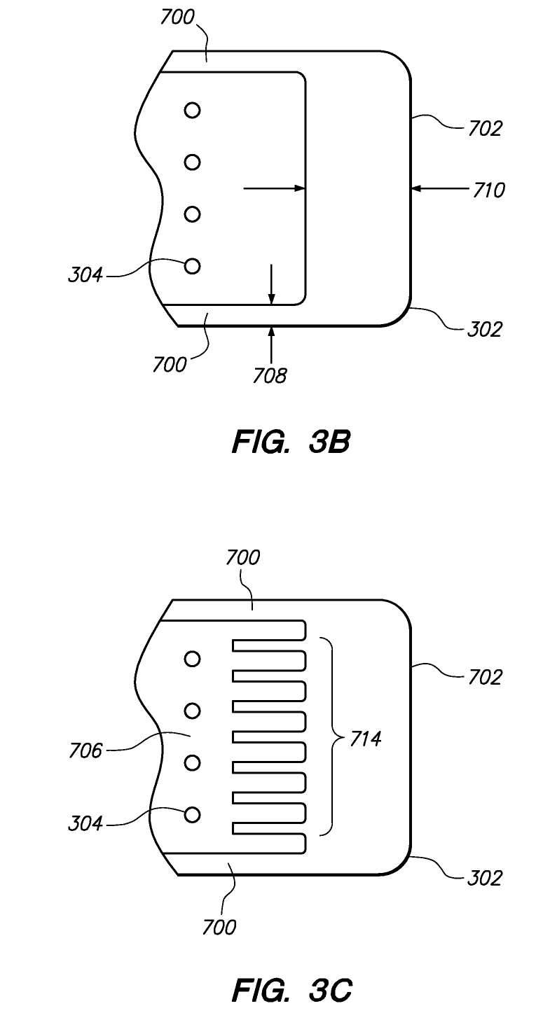

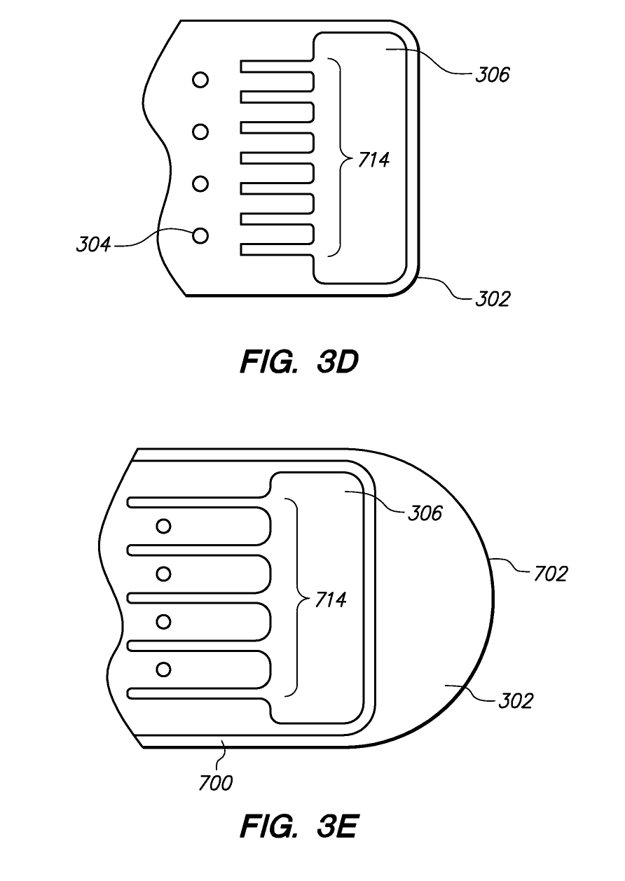

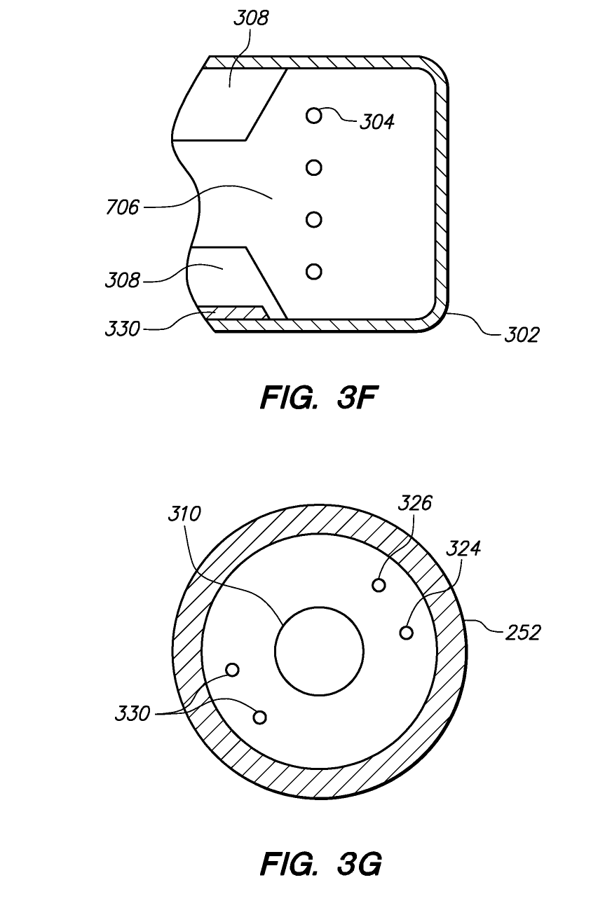

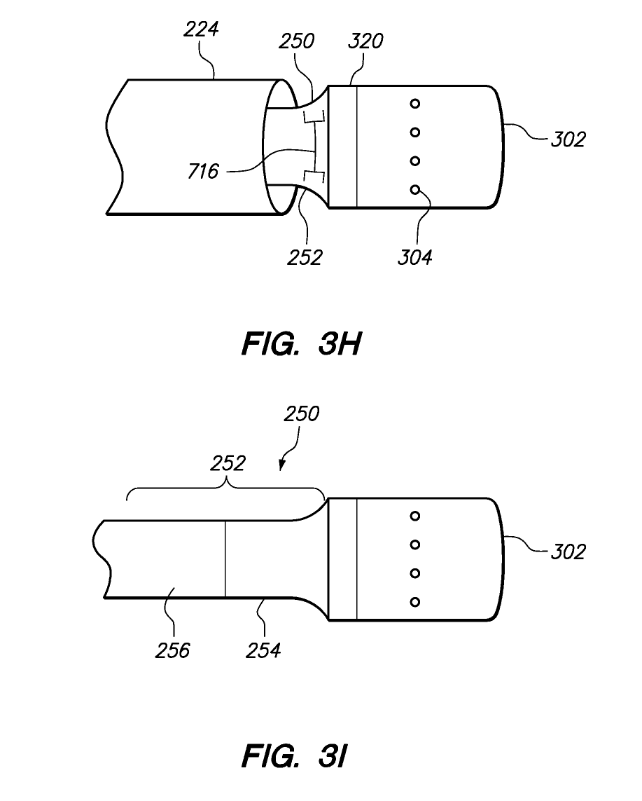

Referring to FIG. 2A, one embodiment of a robotically controlled catheter system (200) is depicted, wherein the elongate aspects of the assembly all are generally configured to have substantially reduced cross sectional profiles. For example, in one embodiment the maximum overall outer diameter of the assembled instrument set is about 7 to about 7.5 French. Similar to that depicted in FIG. 1D, a robotically controlled catheter system (200) includes a first robotic sheath instrument (214) which may be placed into or disposed through, e.g., in a coaxial manner through a working lumen, a second robotic sheath instrument (234). The second robotic sheath (234) may have a tubular body (238) which includes a proximal end (240), a distal end (242), and a “working lumen” or “through lumen” (244) that is appropriately sized to accept the robotic sheath instrument (214), which generally has a smaller tubular body (224). The tubular bodies may have cross sectional shapes that are substantially circular, rectangular, and elliptical or any suitable geometrical shape. The second robotic sheath instrument (234) also has a proximal interface assembly (236) for mechanically interfacing with an electromechanical robotic instrument driver (not shown), such as those described in detail in the aforementioned applications incorporated by reference, to drive or steer the second sheath instrument (234), such as by causing controlled bending of portions of the tubular body (238). Similarly, the first robotic sheath instrument (214) may have a tubular body (224) that is appropriately sized such that the tubular body (224) may be inserted into the through lumen (244) of the second robotic sheath instrument (234). The first robotic sheath instrument (214) includes a proximal end (220), a distal end (222), and a through lumen (218) of its own, and this first robotic sheath instrument (214) may also be robotically steerable through operative coupling with the electromechanical instrument driver. Elongate medical instrumentation, such as an ablation catheter, an irrigated ablation catheter, a guidewire, a needle, or the like, may be passed through the lumen (218) and used to perform various diagnostic or interventional procedures. To facilitate electromechanical steerability of portions of the first robotic sheath instrument (214), such as portions of the tubular body (224), such instrument (214) may also have a proximal interface assembly (216) similar to that (236) for the second robotic sheath instrument (234). The electromechanical, or robotic, instrument driver may be configured to drive or steer the first and second sheath instruments (214, 234) separately or in concert.

A specialized ablation catheter (250) is depicted in FIG. 2A positioned through the working lumen (218) of the first robotic sheath instrument (214). The depicted ablation catheter (250) is similar to that described in reference to FIG. 1E, with the exception that all but the distal portion of such ablation catheter (250) has what is referred to herein as a “necked down” geometric configuration, wherein cross sectional shape is minimized, or necked down, along a portion of its length from a larger shape to a smaller shape (i.e., such as a smaller diameter) to allow for a more slender robotic sheath (214) to engage the necked down portions, while retaining a more conventionally-sized distal shape factor, which may be preferred for clinical reasons. In one embodiment, the distal portion of the necked-down ablation catheter is unable to fit through the working lumen (218) of the first robotic sheath (214). Indeed, the overall diameter of the distal portion may be approximately the same as the outer diameter of the first robotic sheath (214), or approximately the same as the outer diameter of the second robotic sheath (234). With a necked-down proximal ablation catheter geometry, a smaller first robotic sheath may be utilized, along with a smaller second robotic sheath, resulting in a decreased overall shape factor for the elongate assembly. With this decreased profile, the smaller working components may be advanced and steered into smaller pathways or vasculatures of a patient to access targeted sites.

In embodiments wherein the outer diameter is configured to be larger than the inner diameter of the working lumen of the first robotic sheath (214), a specialized assembly configuration may be utilized to engage the various elongate instruments (250, 214, 134). The process of inserting, placing, or disposing a necked-down irrigated ablation catheter (250) through the catheter system (200) may involve “backloading” the surgical instrument to the first robotic sheath instrument (214), and then inserting the first robotic sheath instrument (214) with the surgical instrument through the lumen (244) of the second robotic sheath instrument (234). For clarification, backloading might involve inserting the proximal end of the necked-down catheter (250) into the distal end (222) of the working lumen (218) of the first robotic sheath (214), and advancing such proximal end proximally through the working lumen (218) until it reaches a position adjacent the proximal end of the first robotic sheath (214) where it may be operatively coupled to other components of the system, such as irrigation sources or circuits and/or electrical systems, such as RF generators, thermocouple systems, and/or localization systems. In certain applications it may be desirable to utilize more than one instrument through the working lumen (218) of the first robotic sheath (214) during a given clinical procedure to perform diagnostic and/or interventional procedures. For example, to perform an electrophysiologic ablation procedure within the left atrium of the heart, a trans-septal needle system may be used with the first and second robotic sheath instrument (214, 234) to cross the inter-atrial septum and gain access to the left atrium. After crossing the septum, the trans-septal needle system may be retracted from the heart and body of the patient, and the first robotic sheath instrument (214) may be removed from the instrument driver to backload an ablation catheter onto the first robotic sheath instrument. A first robotic sheath instrument (214) with an ablation catheter may then be inserted into the second robotic sheath instrument (234) as well as attached onto the instrument driver, such that the ablation catheter may be steered and advanced into the left atrium of the heart to perform ablation procedures.

Still referring to FIG. 2A, the necked-down ablation catheter (250) may be operatively coupled to a homeostatic valve assembly and bellow connector (260), a dither clamp (262), a catheter holder clamp (264) to further secure the catheter (250) to the instrument driver (not shown), a clamp connector (266), and a Luer fitting connector (268). As will be discussed in further detail, in the depicted embodiment, the clamp connector (266) is operatively coupled, via a electrical connection, to an RF energy generator and controller (271) enabling the irrigated ablation catheter (250) to perform as well as monitor various aspects of electrophysiologic procedures, e.g., electrical sensing or ablation of tissue structures, and the clamp connector (266) is also operatively coupled, via a fluid connection, by way of a Luer fitting or connector (268), to a pump (270) enabling the catheter (250) to perform irrigated operations using coolant, such as saline or any suitable fluid. The homeostatic valve assembly and bellow connector (260) and the dither clamp (262) may be coupled to a dithering mechanism configured to oscillate, or “dither”, the necked-down catheter (250) relative to the first robotic sheath (214) and facilitate the measurement of forces imparted upon the distal aspect of the ablation catheter (250) by adjacent structures, such as tissue structures, as described in detail in the aforementioned incorporated by reference disclosures, including 11/678,001 and 11/678,016, wherein the notion of dithering one instrument relative to another to facilitate relative load measurement during a period of kinetic friction, as opposed to static friction, between the instruments is described. One embodiment of a process work flow for integrating the necked-down catheter (250) as part of an instrument system (200) comprising one or more robotic sheaths, such as those available under the Artisan® tradename from Hansen Medical, Inc., of Mountain View, Calif., for a clinical procedure is illustrated in FIG. 2B.

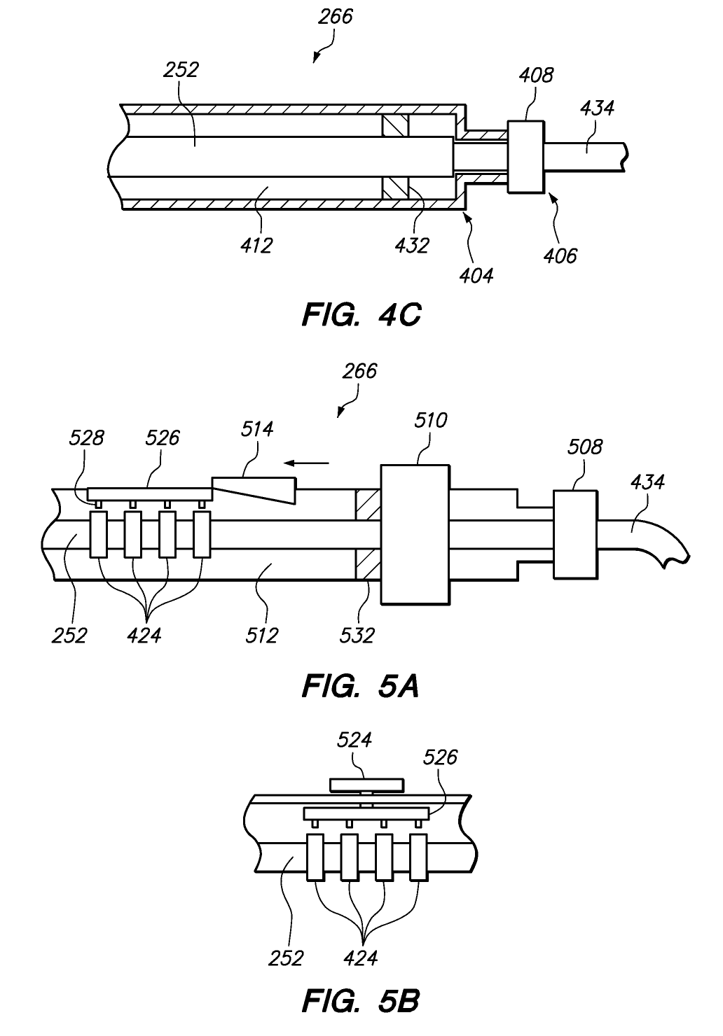

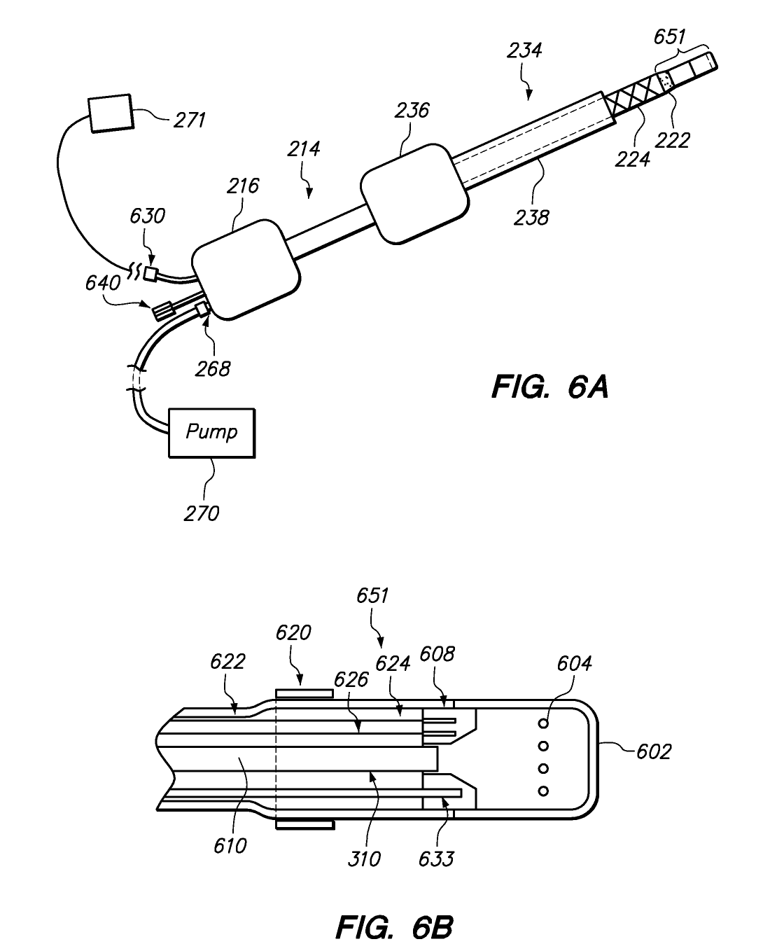

Referring to FIG. 2B, a necked-down catheter is backloaded into a first robotic sheath instrument, as indicated in Step 2021; in an embodiment featuring a proximal clamp for engaging the proximal end of the necked-down catheter, as described below in reference to FIGS. 4A-5B, for example, the proximal end of the necked-down catheter is inserted proximally through the first robotic sheath working lumen until it hits a mechanical stop, such as one provided by the engagement of the distal tip of the first robotic sheath with the relatively oversized distal portion of the necked-down ablation catheter; in such a stopped configuration, connectors featured on the proximal aspect of the ablation catheter may be placed into electrical operative coupling with external systems, such as irrigation pumps and reservoirs, RF energy generators, and the like, by compressing an operation lever (410) or by loosening a homeostatic valve assembly connector knob (510) of a clamp connector (266)), as indicated in Step 2022; releasing the operation lever (410) or tightening homeostatic valve assembly knob (510) may be utilized to create an irrigation seal (as provided by clamp connector (266)), as indicated in Step 2023. An electrical connector may specifically be electrically coupled to an RF generator (such as generator (271) in the system depicted in FIG. 2A), as indicated in Step 2024; and a Luer fitting (268) connection may be established to engage an irrigation tube set, as indicated in Step 2025. Subsequently the catheter (250) may be flushed, as indicated in Step 2026.

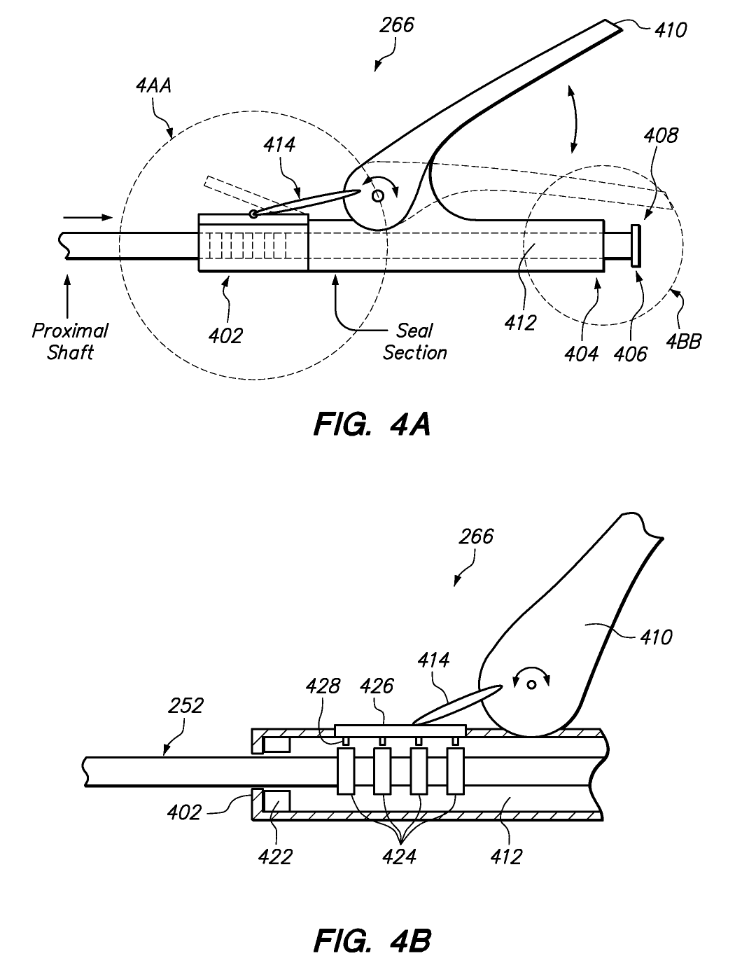

FIG. 3A illustrates one embodiment of a distal tip portion (251) of a necked-down irrigated ablation catheter (250). In this embodiment, the catheter (250) includes an open-loop cooling system to cool the tip of the catheter while ablation is performed. Experiments have shown that a proper lesion is more likely to be formed on the tissue that is being ablated when the temperature of the ablation tip is controlled by cooling. To control or maintain the ablation tip within a desired range of temperatures, an open-loop cooling system may be utilized to transfer heat away from the immediate area of contact between a distal ablation tip and a targeted tissue structure portion. The open-loop cooling configuration may maintain a certain amount of fluid in the tip portion (251) to cool the tip by conduction through a reservoir (706) defined within the distal ablation tip housing (302), the reservoir (706) defined as a volume bounded by the side walls (700) of the distal tip, the distal face surface (702), and the proximal aspect (704) of the distal tip housing (302), which may include access to one or more irrigation lumens (310). Open-loop cooling configurations also are configured to transfer heat through the dispense of a certain amount of fluid through side holes or side ports (304) defined into the tip housing (302). These ports or holes may be circular, oval, or slit-shaped. Such exiting fluid generally will carry heat away from the area, simply by exiting the immediate region when heated, or by exiting the ports (304), gathering additional heat from the region immediately around the interface between the distal tip housing (302) and the targeted tissue, and being free to flow away and disburse heat, or in some circumstances, absorb energy through the latent heat of evaporization of the fluid when vaporized in small volumes. Depending upon the nearby pertinent fluid dynamics, a localized pool of fluid may gather at the ablation site to maintain an appropriate temperature or cool the tip portion (251) of the ablation catheter (250) as well as the tissue that is being ablated. As may be appreciated, ablation is done by passing current from an electrode, such as the tip housing (302) to the tissue that is being ablated. As tissue has certain amount of resistance to current, heat is generated as current is passed into the tissue and generated heat causes lesion to form. If the electrode or tip housing (302) is not cooled sufficiently, the hot electrode or tip housing will cause surface lesion that result in excessive heating of the surface tissue which may cause charring and popping result in undesirable outcomes.

Referring again to FIG. 3A, the irrigated ablation catheter (250) includes a lumen or tube (310) which delivers fluid, such as saline, from an external pump (270 in FIG. 2A) to the tip housing (320) for cooling the tip during ablation procedures. The depicted tip portion (251) embodiment of the catheter (250) also includes a ring electrode or sensor (320) for sensing tissue electrical conductivity either by itself, as in mono-polar sensing, or along with the tip housing (302), as in bipolar sensing, to monitor the ablation procedure and check the condition of the lesion on tissue that is being ablated. The ring sensor (320) may be made of a metallic material or alloy, e.g., platinum and iridium, typical composition may be 90 percent platinum and 10 percent iridium. An electrical wire (322) connects the ring sensor (320) to the generator and controller (271) for controlling and monitoring the sensing operation.

Still referring to FIG. 3A, the depicted tip housing (302) may be referred to as having a “thin shell” design, wherein the wall thickness (708) of the side walls (708) is relatively small compared to the overall diameter (712) of the distal tip. For example, in one embodiment, the side wall thickness (708) dimension may be less than four times smaller than the diameter (712) dimension. Thin walls are desirable in some embodiments because they enhance the ability of nearby fluids, such as saline, to transfer heat away from metal materials comprising the thin walls; less metal and more fluid (for example, in a reservoir) may assist in the overall tip having a greater ability to transfer heat away from a nearby ablative lesion; indeed, in such embodiments, the ratio of water volume to metal volume in the tip is an indicator of heat transfer capability. In one embodiment, the distal face (702) may have a thickness (710) similar to that of the side walls (700), while in other embodiments, as described below, the distal face (702) may have significantly different geometry. The depicted distal tip housing includes holes or ports (304) for dispensing a fluid, such as saline. The tip housing (302) may have any number of holes or ports (304); preferably, the tip housing may have five holes or seven holes. In addition, the holes or ports (304) may be arranged in any arrangement or patterns on the tip housing (302). The holes may be arranged in an aligned pattern as illustrated in FIG. 3A. In addition, the holes may be arranged in a misaligned or staggered or zigzagged pattern.

As briefly described above, to enhance thermal conductivity and heat transfer, the wall thickness of the distal face (702) of the tip housing (302) may be thicker than the wall thickness (708) of the side walls (700), such as two or more times thicker in one embodiment, as illustrated in the embodiment in FIG. 3B. In addition, as depicted in FIG. 3C, the interior surface of the distal face (702) of the distal tip housing (302) may include fins or channels (714) configured to increase surface area engagement between the material comprising the distal face and fluid which may reside in or be circulating or flowing through the adjacent reservoir (706) to further enhance thermal conductivity and heat transfer. In one embodiment, an insert or heat sink member (306) may be added to the tip housing (302) to increase the thermal mass to enhance conductivity and heat transfer; this member (306) may comprise a conductive metal and be geometrically configured with channels or fins (714) to increase surface engagement with nearby fluids, as depicted in FIG. 3D, or may comprise a smoother and more uniform mass without such channels or fins. FIG. 3E further illustrates that the channels or fins (714) may be substantially prominent to provide significant conductivity and heat transfer.

The side walls of the tip housing are substantially cylindrical in one embodiment, with the distal face comprising a substantially planar circular surface face to such cylindrical sidewall configuration, such as in the embodiment depicted in FIG. 3D. In another embodiment, a substantially cylindrical sidewall configuration may be mated with a substantially hemispherically-shaped distal face configuration, as depicted in FIG. 3E, or a half-football or bullet-shaped distal face (not shown). The side walls may also comprise a noncylindrical shape, and the distal face joined to match. The tip housing (302) may be made of a metallic material or alloy, e.g., platinum and iridium, typical composition may be 90 percent platinum and 10 percent iridium. Similarly, the insert or heat sink member (306) may be also made of a metallic material or alloy, e.g., platinum and iridium, typical composition may be 90 percent platinum and 10 percent iridium or the insert (306) made be made of any material suitable for enhancing heat transfer.

Referring back to FIG. 3A, the tip housing (302) may be coupled to the tip portion (251) of the catheter (250) by being secured to the lumen insert (308). The tip housing (302) may be secured to the lumen insert (308) by soldering, welding, fastening by screws, crimping or any suitable means to securely attach the tip housing (302) to the tip portion (251) of the catheter (250). The lumen insert (308) has rounded or chamfered edge as illustrated in FIG. 3A, so as to substantially eliminate or reduce eddy current inside the chamber, cavity, or reservoir (706) provided by the tip housing (302). The lumen insert (308) may be made from a metallic material, e.g., stainless steel, such as stainless steel 303 or 304, such that the lumen insert (308) and tip housing (302) are in electrical communication. Electrical wire (324) is coupled to the lumen insert (308) connecting the lumen insert and tip housing (302) to the generator and controller (271), such that the tip housing may be energized to perform ablation or electrical sensing of tissue structures. In addition, a safety cable or wire (326) may be attached to the lumen insert (308) to act as a tether or leash to the lumen insert and tip housing subassembly. The distal end of the safety cable (326) is secured to the lumen insert (308), while the proximal end of the safety cable may be secured near the proximal portion of the catheter body (252). The catheter (250) may include a thermocouple (330) that may be coupled or potted to the lumen insert (308) to monitor the temperature of the lumen insert and tip housing (302) subassembly. As illustrated in FIG. 3A, in one embodiment, the thermocouple (330) may be potted in the lumen insert (308). In another embodiment, the thermocouple (330) may be potted at the location that is substantially sandwiched between the lumen insert (308) and the tip housing (302) as illustrated in FIG. 3F.

FIG. 3G illustrates a cross sectional view of the irrigated ablation catheter (250). As illustrated, the catheter includes a tubular body (252), a lumen or tube (310) for delivering coolant, such as saline, electrical wire (324), safety cable or leash (326), and thermocouple wires (330). Indeed, the necked-down ablation catheter may have one or a plurality of electrical leads coupled between a distally-located temperature sensing device in the distal ablation tip and the proximal portion of the catheter. Suitable temperature sensing devices include but are not limited to thermocouples, infra-red sensors, and microwave radiometers. Although FIG. 3G illustrates the tubular body (252) to have a substantially round or circular cross section, the tubular body may have any suitable cross sectional shape, e.g., square, rectangle, elliptical, oval, star, pentagon, octagon, etc.

FIG. 3H illustrates an embodiment of a necked-down ablation catheter (250) in connection with the tubular body (224) of the first robotic sheath instrument (214). The transition geometry (714) between the distal ablation tip (302) outer shape and the distal aspect of the tubular body may be tapered, as in the transition geometry (714) of the depicted embodiment, or stepwise (not shown), wherein an abrupt step is defined between the two shapes. As described above, since the catheter (250) has a necked-down body, the robotic instrument that is used to steer the catheter (250) may be made smaller, in particular the tubular body of the robotic sheath instrument, such that the combination may access greater range of body pathways or vasculature due to its reduced size. For example, the tip portion (251) may have a diameter of approximately about 7 French to approximately about 7.5 French, while the necked-down body has diameter of approximately about 6 French, whereas the diameter of a conventional non-necked-down catheter may have a proximal body diameter of approximately 8 French or larger, thus necessitating a larger sheath around such body for sheath-based navigation.

FIG. 3I illustrates that distal portion of the tubular body (252) of the necked-down catheter (250) may be comprised of sections having different stillness or flexibility. For example, the first section (254) may have a certain stillness that promotes articulation, while the section (256) may have a different stillness that promotes dithering motion in the axial direction. The first section (254) may have a durometer stiffness of about 40 D, while the second section (256) may have a durometer stiffness of about 70 D. The first and second sections (254, 256) may be made from tubes with different durometer stiffness. In addition, the first and second sections (254, 256) not be made of tubes with different durometer stiffness, instead the difference in stiffness may be achieved by using different braiding patterns, tightness, spacing, pitch, etc. to the different stiffness characteristics.

Referring to FIG. 4A, one embodiment of a clamp connector (266) for operatively coupling, in an efficient and reliable manner, the proximal portion of a necked-down ablation catheter to other related structures subsequent to positioning such proximal portion through a working lumen of a steerable sheath, as described above in reference to FIG. 2A, for example, is illustrated in more detail. As shown in FIG. 4A, the clamp connector (266) includes a front receiving port (402), lumen 412, operation lever (410), lever arm (414), back stop (404), Luer fitting (408), and a rear receiving port (406). The front receiving port (402) is configured to securely receive the proximal end of the catheter body (252) of the necked-down irrigated ablation catheter (250). In a normal inactivated state, the front receiving port (406) may be closed or block by a sealing member (not shown). Opening and closing of the sealing member is controlled by the operation lever (410). The operation lever (410) is spring loaded, e.g., a torsion spring, compression spring, etc. In a neutral state, the spring action of the operation lever (410) maintains the sealing member in a closed condition; while in an activated state, e.g., with the operation lever (410) being compressed, the operation lever (410) maintains the sealing member in an opened condition. As indicated by the work flow process illustrated in the process flow chart of FIG. 2B, with the sealing member in an open state, the proximal end of the catheter body (252) is inserted into the front receiving port (402) through the lumen (412) until it hits dead stop (404) of the clamp connector (266). To ensure that the catheter body (252) is proper inserted into the clamp connector (266), an indicator marker (not shown) may be provided to verify that the catheter body (252) is properly inserted all the way to the dead stop (404) in addition to the physical feedback from hitting the dead stop (404). FIG. 4B illustrates the close-up view 4AA to show the front portion of the clamp connector (266) in more detail. The front receiving port (402) is appropriately sized to receive the catheter body (252). To provide sealing and snug fit, the clamp connector (266) includes a front seal (422). The front seal (22) provides a tight seal against the catheter body (252), and as the catheter body is inserted and slid into the opening of the front receiving end into the lumen (412), the front seal (422) also acts as a wiper and wipes off or squeegees off any fluid or moisture on the catheter body (252). As illustrated in this close-up view, the proximal end of the catheter body (252) includes conductor rings (424), wherein each of the conductor rings is electrically coupled to each one of the respective conductors (ring wire (322), electrical wire (324), and thermocouple wires (330)) located near the distal portion of the catheter body (252) for providing power, control, monitoring, etc. to various devices (ablation electrode (302), ring sensor (320), thermocouple (330)). In this example, four conductor rings are illustrated, because there are four conductors that require power, control, or monitoring. In other embodiments there may be less or more conductor rings depending on the number of electrical connections are needed to provide power, monitoring, control, etc. that are required for operating the equipment, apparatuses, or devices at the distal portion of the catheter. It is important to note that each of these substructures comprising the proximal aspect of the necked-down ablation catheter, and generally the overall outer shape profile of the proximal aspect of the necked-down ablation catheter, is configured to be slidably engaged and threaded through a steerable sheath to which is may be operably coupled, as described in reference to FIG. 2A. Such an assembly configuration increases the importance of providing a removable quick-connect hardware configuration facilitating a low-profile necked-down catheter proximal end configuration during assembly, which efficient conversion to an integrated assembly having reliable connectivity between various contacts and structures.

Still referring to FIG. 4B, once the proximal portion of the catheter body is properly inserted into the clamp connector (266), the operation lever (410) is released which in turn activates the lever arm (414) and the contact pad (426), wherein the contact teeth (428) engage the conductor rings (424) and the electrical circuits from the generator and controller (271) to the various apparatuses, such as the electrode (302), ring sensor (320), and thermocouple (330) are complete. In some embodiments, the conductor rings (424) are protected by a membrane to insulate or protect the conductor rings from exposure to moisture, oils, dirt, etc. When the contact pad (426) is activated, the contact teeth (428) penetrate, puncture, tear, rip, etc. the membrane to contact the conductor rings (424) to complete the respective electrical circuits. In some embodiment, the membrane may stay intact and electrical connection may be made through the membrane barrier.

FIG. 4C illustrates the close-up view 4BB to show the rear portion of the clamp connector (266) in more detail. As illustrated, the proximal portion of the catheter body (252) is inserted into the clamp connector (266) through the lumen (412) hitting the back stop (404). An additional seal member (432) provides additional seal and support to the catheter body (252). A coolant hose (434) is inserted through the Luer fitting at the rear receiving port (406), which provides a tight fluid seal against the coolant hose. At this point of installation in this embodiment, the open-loop cooling system is complete. Coolant, such as saline, is provided or pumped from pump (270), delivered by the coolant hose (434) to the clamp connector (266) and to the proximal end of the catheter body (252); thus providing coolant to the irrigated ablation catheter (250).

FIG. 5A illustrates an alternate embodiment of the clamp connector (266). For this embodiment, the port to receive the catheter body (252) may be opened or closed by a turnable knob (510) rather than a lever (410). With such embodiment, the catheter body (252) may be inserted into the clamp connector (266) through the lumen (512) and into an elastomeric seal (not shown) when the turn knob (510) has been activated to open the elastomeric seal to receive the catheter body (252). The turn knob (510) may be adjusted to ensure the elastomeric seal fits tightly around and against the catheter body (252). In some embodiments of this clamp connector (266), a back stop is also provided to ensure the catheter body (252) is installed and positioned properly. In addition, an indication marker may be provided on the catheter body (252) to indicate and ensure that the proximal end of the catheter body has been inserted sufficiently into the clamp connector (266). An O-ring (532) may also be provided to additionally position and sealing the catheter body (252). To complete the electrical circuit from the generator and controller (271), the activation button (514) is pushed into position to engage the contact pad (526) with the conductor rings (424) by means of the contact teeth (526). In another embodiment, as illustrated in FIG. 5B, instead of the activation button (514) that is pushed into position to engage the contact pad (526), a screw-drive type button (524) may be used to either advance or retract the contact pad (526) to engage the contact teeth (526) with the conductor rings (424). As discussed previously, the conductor rings (424) may be protected or sealed by a membrane to protect the rings from water, moisture, oils, dust, dirt, etc. This membrane may be penetrated, punctured, torn, ripped, deformed, etc. by the contact teeth (526) to complete the electrical circuit with the generator and controller (271). In some embodiments, the membrane may be kept intact and electrical connection may be made through the membrane. To complete fluid connection from the coolant pump (270) to the catheter (250), a coolant hose (434) configured to deliver coolant, such as saline, may be inserted into the clamp connector (266) by way of the Luer fitting (508). The Luer fitting (508) is configured to ensure a fluid tight seal around the coolant hose (434). With the connection of the coolant hose (434) and the catheter body (252) in the clamp connector (266), the open-loop cooling system of the irrigated ablation catheter (250) is complete.

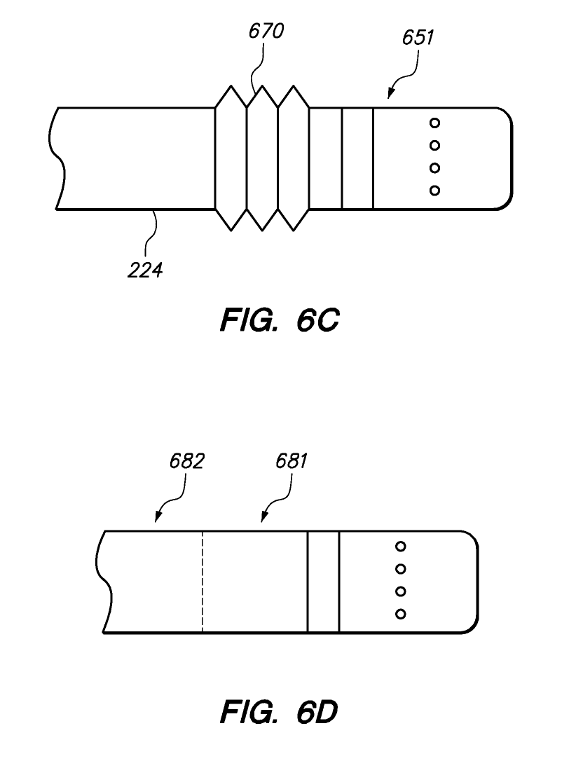

FIG. 6A illustrates another embodiment of a robotically controlled system with a working instrument (such as a conventional ablation catheter, an irrigated ablation catheter, etc.) integrated directly on a robotic sheath instrument. In this example, an irrigated ablation module (651) substantially similar to the tip portion (251) of the irrigated ablation catheter (250) illustrated and disclosed in FIG. 3A. The irrigated ablation module (651) secured to the robotically controlled sheath (224) by various conventional means, such as thermally fusing the proximal end of the irrigated ablation module (651) to the distal end (222) of the robotically controlled sheath (224). A material sold under the tradename “Pebax” is an example of a material that may be used to thermally bond or fuse the ablation module (651) to the robotic sheath (224). FIG. 6B illustrates a cross sectional view of the irrigated ablation module (651). The irrigated ablation catheter module (651) may connect with a lumen or tube (610) from the robotic sheath (224) from which coolant or fluid is delivered, such as saline, from the pump (270) to the irrigated ablation module (651) for cooling the module (651) during ablation procedures. The module (651) also includes a ring electrode or sensor (620) for sensing tissue electrical conductivity either by itself, in mono-polar mode, or along with the tip housing (302), in bipolar mode, to monitor the ablation procedure and check the condition of the lesion on tissue that is being ablated. The ring sensor (620) may be made of a metallic material or alloy, e.g., platinum and iridium, typical composition may be 90 percent platinum and 10 percent iridium. An electrical wire (622) connects the ring sensor (620) to an electrical connector (630), generally located at the proximal portion of the robotic sheath instrument, to the generator and controller (271), as illustrated in FIG. 6A, for controlling and monitoring the sensing operation.

Still referring to FIG. 6B, the module (651) includes a tip housing (602), wherein the tip housing includes holes or ports (604) for dispensing a fluid, such as saline. The tip housing (602) may have any number of holes or ports (604); preferably, the tip housing may have five holes or seven holes. In addition, the holes or ports (604) may be arranged in any arrangement or patterns on the tip housing (602). The holes may be arranged in an aligned pattern as illustrated in FIG. 6B. In addition, the holes may be arranged in a misaligned or staggered or zigzagged pattern. To enhance thermal conductivity and heat transfer, the wall of the tip housing (602) may be thicker at the tip similar to the illustration shown in FIG. 3B. In addition, the wall of the tip housing (602) may include fins to further enhance thermal conductivity and heat transfer similar to the illustration shown in FIG. 3C. Alternatively, an insert (not shown) may be added to the tip housing (602) to increase the thermal mass to enhance conductivity and heat transfer. Similar to the illustration shown in FIG. 3D, the insert may include fins to further enhance conductivity and heat transfer or it may simply be a block of material without fins. Similar to the illustration shown in FIG. 3E the fins may be substantially prominent to provide significant conductivity and heat transfer. In addition, the tip housing (602) may have a substantially blunt tip or block or rectangular profile shaped tip or the tip housing (602) may have a substantially round or hemispherical shaped tip. The tip housing (602) may be made of a metallic material or alloy, e.g., platinum and iridium, typical composition may be 90 percent platinum and 10 percent iridium. Similarly, the insert may be also made of a metallic material or alloy, e.g., platinum and iridium, typical composition may be 90 percent platinum and 10 percent iridium or the insert made be made of any material suitable for enhancing heat transfer.

Still referring to FIG. 6B, the tip housing (602) may be coupled to the tip portion of the module (651) by being secured to a lumen insert (608). The tip housing (602) may be secured to the lumen insert (608) by soldering, welding, fastening by screws, crimping or any suitable means to securely attach the tip housing (602) to the tip portion of the module (251). The lumen insert (608) may have rounded or chamfered edge as illustrated in FIG. 6B, so as to substantially eliminate or reduce turbulent flow of coolant into the chamber or cavity provided by the tip housing (602). The lumen insert (608) may be made from a metallic material, e.g., stainless steel, such as stainless steel 303 or 304, such that the lumen insert (608) and tip housing (602) are in electrical communication. Electrical wire (624) is coupled to the lumen insert (608) connecting the lumen insert and tip housing (602) via the electrical connector (630) to the generator and controller (271), such that the tip housing may be energized to perform ablation or electrical sensing of tissue structures. In addition, a safety cable or wire (326) may be attached to the lumen insert (608) to act as a tether or leash to the lumen insert and tip housing subassembly. The distal end of the safety cable (626) is secured to the lumen insert (608), while the proximal end of the safety cable may be secured and mechanically associated with the dithering mechanism (640) near the proximal portion of the robotic sheath instrument (214). The safety cable (626) may be used to dither the irrigate ablation module (651) for force sensing as well as other purposes as described in the aforementioned disclosure incorporated into this description.

FIG. 6C illustrates one embodiment in which a bellow (670) is incorporated to the module (651) and the distal tip (222) of the sheath body (224) to facilitate oscillatory motion, or “dithering”, of the module relative to other structures, to, for example, facilitate distal force sensing, as described in detail in the aforementioned incorporated by reference disclosures. In addition, the sheath body (224) may be constructed such that it has sections with different stillness or flexibility; preferably near the distal portion of the body. For example, as illustrated in FIG. 6C, a first section (681) may have a certain stillness that promotes articulation, while the second section (682) may have a different stillness that promotes dithering motion in the axial direction. The first section (681) may have a durometer stiffness of about 40 D, while the second section (682) may have a durometer stiffness of about 70 D. The first and second sections (681, 682) may be made from tubes with different durometer stiffness. In addition, the first and second sections (681, 682) not be made of tubes with different durometer stiffness, instead the difference in stiffness may be achieved by using different braiding patterns, tightness, spacing, pitch, etc. to obtain the different stiffness characteristics.

Referring back to FIG. 6B, the ablation module (651) may include a thermocouple (633) that may be coupled or potted to the lumen insert (608) to monitor the temperature of the lumen insert (608) and tip housing (602) subassembly. As illustrated in FIG. 6B, in one embodiment, the thermocouple (633) may be potted in the lumen insert (308). In another embodiment, the thermocouple (633) may be potted at the location that is substantially sandwiched between the lumen insert and the tip housing similar to the illustration shown in FIG. 3F. All electrical conductors, such as electrical wire (624), ring wire (622), and thermocouple wires (633), are all route to the proximal portion of the robotic sheath instrument (214) and connect or couple with the electrical connector (630) and further connected to the generator and controller (271) to power, control, monitor, etc. all electrical operations, such as ablation, sensing, etc.

Still referring to FIG. 6B, at the proximal portion of the robotic instrument (214) a coolant hose is connected to the robotic instrument to provide coolant for an open-loop cooling system via a Luer fitting (640). By incorporating the irrigated ablation module (651) directly to the robotic sheath instrument (214), significant amount of hardware may not be necessary to enable the functionality of this integrated irrigated ablation catheter. Accordingly, the size of the robotic sheath instrument can be made smaller to enhance greater or improved access to various vasculatures and tissue structures.

As described above, a dithering mechanism (640) may be utilized to measure and/or calculate forces applied upon the distal portion of a necked-down ablation catheter positioned through a sheath instrument. For example, such a configuration may be utilized to measure forces imparted to the distal ablation tip by adjacent tissues, such as endocardial tissues that may be the subject of an ablation, mapping, or other endocardial procedure, as the ablation tip is advanced toward and into contact with such tissues. Further details of such a measurement configuration for coaxially and slidably associated instruments is disclosed in the aforementioned incorporated by reference disclosures, including 11/678,001 and 11/678,016.

While multiple embodiments and variations of the many aspects of the invention have been disclosed and described herein, such disclosure is provided for purposes of illustration only. For example, wherein methods and steps described above indicate certain events occurring in certain order, those of ordinary skill in the art having the benefit of this disclosure would recognize that the ordering of certain steps may be modified and that such modifications are in accordance with the variations of this invention. Additionally, certain of the steps may be performed concurrently in a parallel process when possible, as well as performed sequentially. Accordingly, embodiments are intended to exemplify alternatives, modifications, and equivalents that may fall within the scope of the claims.

1. An ablation instrument system, comprising:
a. a robotically-steerable sheath having proximal and distal ends and defining a working lumen between said ends; and
b. a necked-down ablation catheter having a distal ablation tip, a proximal end, and a tubular body coupling the distal ablation tip and the proximal end;
wherein at least a portion of the tubular body is slideably disposed through the working lumen of the robotically-steerable sheath; and wherein the distal ablation tip has an outer diameter configured to prevent such tip from fitting into the working lumen of the sheath.

2. The instrument system of claim 1, wherein the distal ablation tip and tubular body are coupled with a substantially stepwise neckdown.

3. The instrument system of claim 2, wherein the distal ablation tip has a substantially cylindrical side outer shape with a substantially flat distal face.

4. The instrument system of claim 2, wherein the distal ablation tip has a substantially cylindrical side outer shape with a substantially hemispherical distal face.

5. The instrument system of claim 2, wherein the distal ablation tip has a substantially spherical outer shape.

6. The instrument system of claim 2, wherein the distal ablation tip has a substantially cylindrical side outer shape with a substantially bullet-shaped distal face.

7. The instrument system of claim 1, wherein the ablation catheter comprises a first irrigation lumen defined between the proximal end and the distal tip.

8. The instrument system of claim 7, wherein the distal tip comprises thin-shell design comprising sidewalls having a sidewall thickness, a distal face having a distal face thickness, and defining a distal irrigation reservoir volume defined between the sidewalls, distal face, and a proximal surface of the distal ablation tip, the irrigation reservoir volume being accessible via the first irrigation lumen.

9. The instrument system of claim 8, wherein the outer diameter of the distal tip is at least four times greater than the sidewall thickness.

10. The instrument system of claim 8, wherein the distal face has a substantially uniform thickness.

11. The instrument system of claim 10, wherein the distal face thickness is at least two times greater than the sidewall thickness.

12. The instrument system of claim 8, wherein the distal face has an inner surface defining a distal portion of the distal irrigation reservoir volume, the distal face inner surface having a channeled geometry to maximize surface area of the distal face inner surface.

13. The instrument system of claim 8, further comprising a metallic heat sink member suspended within the distal irrigation reservoir volume.

14. The instrument system of claim 13, wherein the metallic heat sink member has a surface shape at least partially defining channels configured to maximize surface engagement between the metallic heat sink member and fluids which may be present in the distal irrigation reservoir volume.

15. The instrument system of claim 8, wherein a plurality of sideholes are defined through the sidewalls to allow for the escape of irrigation fluids pressurized into the distal irrigation reservoir volume.

16. The instrument system of claim 8, the ablation catheter further comprising a return irrigation lumen defined between the proximal end and the distal tip and configured to allow at least a portion of fluid pressurized through the first irrigation lumen to the irrigation reservoir volume to return to the proximal end of the ablation catheter.

17. The instrument system of claim 7, wherein the necked-down ablation catheter comprises at least one electrical lead coupled between the distal ablation tip and the proximal end, and a proximal quick connect interface configured to allow coupling of an electrical source to the at least one electrical lead as well as coupling of an irrigation source to the first irrigation lumen with manual actuation of a single mechanical coupling.

18. A method of conducting a minimally invasive ablation procedure, comprising:
a. coupling a necked-down ablation catheter through a working lumen of a robotically-steerable sheath by threading a proximal end of the ablation catheter into the lumen through a distal end of the sheath and continuing to advance the ablation catheter relative to the sheath until the proximal end of the ablation catheter at least partially emerges from a proximal end of the sheath;
b. connecting an RF energy source and an irrigation fluid source to the proximal end of the ablation catheter;
c. coupling at least the robotically-steerable sheath to an electromechanical instrument driver;
d. inserting at least the distal ends of the coupled ablation catheter and robotically-steerable sheath into a patient; and
e. utilizing the electromechanical instrument driver to navigate the distal end of the sheath and thereby navigate the distal end of the ablation catheter.

19. The method of claim 18, wherein connecting an RF energy source and an irrigation fluid source to the proximal end of the ablation catheter comprises actuating a proximal quick connect interface configured to allow coupling of the RF energy source to at least one electrical lead on the ablation catheter as well as coupling of the irrigation fluid source to a first irrigation lumen defined by the ablation catheter, with manual actuation of a single mechanical coupling.

20. The method of claim 18, wherein inserting comprises commanding the electromechanical instrument driver to insert the coupled ablation catheter and steerable sheath.

21. The method of claim 18, further comprising advancing irrigation fluid from a proximally disposed reservoir to a reservoir disposed within the distal end of the ablation catheter.

22. The method of claim 21, further comprising disbursing at least a portion of said irrigation fluid out of the distal end of the ablation catheter through a plurality of side ports.

23. The method of claim 21, further comprising returning at least a portion of the advanced irrigation fluid from the distal end of the ablation catheter to the proximal end of the ablation catheter via a return irrigation lumen.

24. A method, comprising:
a. inserting a distal portion of an elongate instrument into a patient's body, the elongate instrument comprising a necked down ablation catheter having a distal ablation tip, a proximal end, and a tubular body coupling the distal ablation tip and the proximal end, wherein at least a portion of the tubular body is slideably disposed through the working lumen of a robotically-steerable sheath and the outer diameter of the distal ablation tip is greater than the inner diameter of the sheath defining the working lumen; and
b. dithering the elongate instrument relative to the sheath.

25. The method according to claim 24, further comprising:
a. advancing the distal tip of the necked down ablation catheter into contact with a tissue structure surface within the patient's body; and
b. calculating loads imparted to the distal tip of the necked down ablation catheter as a result of such contact.

26. The method according to claim 25, wherein calculating comprises measuring relative loads between the necked down ablation catheter and sheath during the dithering.
